(19) United States
(12) Patent Application Publication
(10) Pub. No.: US 2004/0049197 A1
(43) Pub. Date: Mar. 11, 2004

(54) Dorsolumbar and lumbosacral vertebral fixation system

(75) Inventor: Jose Vicente Barbera Alacreu, Valencia (ES)

Correspondence Address: BIRCH STEWART KOLASCH & BIRCH, PO BOX 747, FALLS CHURCH, VA, 22040-0747, US

(73) Assignees: Jose Vicente Barbera Alacreu; Jose J. Aparici Marin; Jose Ramon Bilbao Ortiz De Zarate

(21) Appl. No.: 10/660,685

(22) Filed: Sep. 12, 2003

Related U.S. Application Data
(62) Division of application No. 09/444,449, filed on Nov. 22, 1999, now abandoned.
Application No. 09/444,449 is a continuation-in-part of application No. 08/970,382, filed on Nov. 14, 1997, now Pat. No. 6,001,102.
Application No. 08/970,382 is a continuation of application No. 08/355,258, filed on Dec. 8, 1994, now abandoned.

(51) Int. Cl. A61B 17/56

(52) U.S. Cl. 606/73

(57) ABSTRACT

The invention relates to a method and a device for transpedicular vertebral attachment comprising the steps of drilling a pedicle with a drill as for example a twist drill in order to form a hole in the pedicle; removing the drill from the hole thus formed in the pedicle; screwing a plug into the hole in the pedicle thus formed by the twist drill; whereby an anchorage of the plug is established in the pedicle. The invention also relates to a device for connecting an attachment rod and a pedicular plug in a transpedicular vertebral attachment system, said device comprising: a generally cylindrical, C-shaped member having two ends which form an opening therebetween, the C-shaped member further having a centrally disposed opening, a ball joint being housed in said centrally disposed opening of the C-shaped member, the attachment rod being housed in the ball joint, the ball joint being in direct engagement with the attachment rod; a pair of hollow cylinders, one of the cylinders being attached to each end of the C-shaped members, a screw being received in both of the hollow cylinders, the screw being received insertable in the pedicular plug; and a means, preferably a nut, for locking the screw in position in the pair of hollow cylinders. Finally, the invention relates to a dorsolumbar and lumbosacral vertebral fixation system, wherein the system consists of one or various connectors or couplings, a rod, a transversal traction device and means of vertebral fixation, with assembly carried out by the attaching the tail of the vertebral element—coupling—rod, the first assembly stage of the system being the introduction of the fixation elements, either to the pedicle or the vertebral laminae, a second stage of the insertion of the rod through the connectors, and a third stage in which the connectors are connected to the tails of the fixation elements by means of locknuts.

### FIELD OF THE INVENTION

The present invention relates to a transpedicular vertebral attachment system. Furthermore, the invention relates to a transpedicular vertebral attachment device. Said device provides various advantages for the intended function of said device, which advantages are in detail described hereinafter and will be apparent to persons skilled in the present field of the art from the subsequent description of the preferred embodiments. In addition, the dorsolumbar and lumbosacral fixation system that is also the object of the present invention consists of an assembly of connectable parts that form a solid and compact system consisting of vertebrae attachment elements that, by means of clamps that are adjustable around a swivel coupled to a rod, can be positioned so that by closing them, using locknuts, the desired fixation can be achieved.

### BACKGROUND OF THE INVENTION

There is currently known the technique regularly used for transpedicular vertebral attachment using for example a 5 cm long screw. This technique suffers from the drawbacks derived from the length and constituition of the screw and of the anchorage site, which may cause rocking of the front end of the screw proper to osteoporotic vertebrae, with the consequent loss of pull-out strength and with the serious risk of extending beyond the vertebra anterior cortical layer with the tip of the screw, possibly perforating viscera or blood vessels.

To date different elements have been used as integral parts of vertebral fixation systems. One of these depends on connectors or couplings which always present a fixation screw at 90° to a rod, in such a way that the angle formed by the coupling axes for the pass of these two parts is straight.

The main disadvantage is that the onset between the screw and the rod is not always at an angle of 90°, as a result of which it is necessary to bend and turn the rod in order to bring the coupling to the screw head, increasing weak points, surgical intervention times, complications etc. At the same time these connectors have a single orifice, i.e. there is no possibility of modifying the position of the rod when there is any difficulty of alignment of the rod in relation to the screw heads, and therefore it is necessary to bend the rod with the problems that are then involved in such operations.

Another of the elements used in current fixation systems, are rods that are fully threaded, in such a way that the connector has two locknuts for fixation, as well as setscrews and nut locking. At the same time, once the threaded rod has been moulded, there are problems in fitting the other parts into place, as well as the fact that the threads become stress-raisers that can lead to breakage and cracking through material fatigue. This moulding also leads to a permanent deformation of the material.

In short, this system leads to a great deal of mechanical complications, due to the number of parts involved, in such a way that the surgical intervention tends to be lengthened, increasing the risks of complications due to dropping or loss of parts, etc.

The pedicle screws that are used are self-threading, i.e., they tap into the vertebral pedicles, with threads that have been conditioned to this end. These screws usually encounter problems of rejection by the bone (pull-out effect), in that the thread worked into the bone, due to the constant movement thereof, can widen the hole which will result in a full or partial pull-out. Also, in cases or osteoporosis, there is no solid bone mass and the thread does not bind to the bone.

In cases of revision, when it may be necessary to remove a screw due to its lack of hold, a larger diameter screw may then be used to replace it, which is strongly advised, given the physical limitations of the vertebra itself, or the fixation is done on the basis of another system. In order for replacement screws to have good fixation they will need two points of contact, these are the two corticals. This means that the tip of the screw will emerge, thus involving the risk of damage to major blood vessels, intestines, rectum, etc., and complications in the intervention.

On the other hand, in conventional dorsolumbar and lumbosacral fixation systems hooks are used which can be screwed directly onto the rod, with the result that the position of the hook determines the pass of the rod, i.e. its direction, thus obliging the rod to be bent to shape, this in turn must have an angle of 90°, with the problems of additional load on the rod, as described above.

Through the use of a dorsolumbar and lumbosacral vertebral fixation system all of these disadvantages can be palliated or avoided. To this end, elements that have been improved, in comparison with existing elements, are used, such as connectors with adjustable orifices incorporating multidirectional swivels between the screws and the rod, as well as fixation screws that are hollow expansion screws to impede them pulling out of the bone.

### SUMMARY OF THE INVENTION

The present inventors have devised improvements in a transpedicular vertebral attachment system of the type describe above, consisting of replacing the traditional length srews with another, much shorter screw applied on a plug of a biocompatible and high stability material which may be, for example a silastic polymer or any other polymer of similar mechanical features or any metal or metal alloy which is inserted into the pedicle and is only slightly longer than the latter. The objective is that, when the screw is screwed into the plug, the front inner end of the latter is widened on being expanded by the screw. Of course, a sufficient attachment may also be achieved by using a plug which is structured in a manner that the front inner end cannot be widened and, nevertheless, provides the desired secure attachment of the system by being inserted into the pedicle.

The invention relates to a method for transpedicular vertebral attachment, comprising the steps of

drilling a pedicle with a drill, e.g. a twist drill, in order to form a hole in the pedicle;

removing the drill from the hole thus formed in the pedicle;

screwing a plug into the hole in the pedicle formed by the drill, the plug having a first end and a second end and having an opening at first end thereof which is not inserted into said hole and having a tubular stem being provided with equidistantly spaced radial slits extending lengthwise along the stem at the second end;

inserting a threaded portion of a screw into the opening of the plug; and

expanding the plug radially by inserting the threaded portion of the screw whereby anchorage of the plug is established in the pedicle.

The invention also relates to a system for transpedicular vertebral attachment, said system comprising:

a drill as, for example, a twist drill for forming a hole in the pedicle;

a plug having a first end and a second end, the first end of the plug having an opening provided therein, the second of the plug being insertable into the hole in the pedicle formed by the drill and having a tubular stem being provided with equidistantly spaced radial slits extending lengthwise along the stem; and

means for expanding the second end of the plug to thereby enlarge the second end to form an anchorage of the plug in the pedicle, said means for expanding comprising a screw which is insertable into the opening in the first end of the plug.

In a preferred embodiment, the plug has a radially widened portion, and the screw has a shoulder, the screw having a threaded portion which extends from an end of the screw to the radial shoulder, the threaded portion of the screw being slightly longer than a length of the pedicle, the radial shoulder of the screw being adapted to engage the radially widened portion of the plug to be able to stop insertion of the screw into the opening of the plug.

In another preferred embodiment, the system of the invention comprises a plug having a first and a second end, the second end of the plug being insertable into the hole in the pedicle formed by a drill and having a tubular stem. Also forming part of the invention are means devised to complete the intersegmentary attachment of the vertebra for joining together the screws inserted into the vertebral pedicles. Such means are formed essentially by rods having special characteristics for an attachment between the anchored levels and by gripper arms also structured especially to form clamps for a fixation of said rods to the pedicular screws with the aid of a multidirectional coupling providing an anchorage of the rods to the pedicular plugs or plug and screw devices.

According to the present invention, there is also provided a device for connecting an attachment rod and a pedicular plug in a transpedicular vertebral attachment system, said device comprising

a generally cylindrical, C-shaped member having two ends which form an opening therebetween, said C-shaped member further having a centrally disposed opening, a ball joint being housed in the centrally disposed opening of the C-shaped member, the attachment rod being housed in the ball joint, the ball joint being in direct engagement with the attachment rod;

a pair of hollow cylinders, one of the cylinders being attached to each end of the C-shaped members, a screw being received in both of the hollow cylinders, the screw being insertable in the pedicular plug; and

a nut for locking the screw in position in the pair of hollow cylinders.

With the system and device according to the present invention, the following purposes are obtained:

(1) A shorter screw, avoiding the danger of perforating viscera or blood vessels, which may happen when the conventional screws are used;

(2) a greater resistance to pull-out forces, since the inner end of the plug is much wider than the outer end (in the case that the extendable plug is used), preventing it from being forced out backwards; likewise, since an exclusively pedicular anchorage is available, this being the strongest part of the vertebra, the possibility of rocking movements of the front end of the screw, proper to osteoporotic vertebra, is avoided.

Thus, the improvements based on the use of a shorter screw and plug include the advantages of the other systems, i.e. usual plugs without extendable second end, which may also be used in accordance with the present invention, while also providing the improvement derived from the facts of applying the forces exclusively on the most solid part of the vertebra, i.e. the pedicle. It also avoids the risk of the pull-out strength being lost in osteoporotic vertebrae, since the inner portion of the plug may be widened to have, in its widened states, a larger diameter at the second end. Finally, with the improvements of the invention, it is not possible to exceed the anterior cortical layer of the vertebra with the screw tip, thereby eliminating the concomitant risks.

Thus, the improvement provide greater security and support, when this technique is used in osteoprotic patients the vertebrae of whom have a considerable lower density of the bone tissue of the vertebral body.

Furthermore, the improvements of the present invention afford the advantages described above, apart from others which will be readily gathered from the embodiments of vertebral attachment system with excellent transpedicular support provided with such improvements, described in further detail hereinafter to facilitate the understanding of the above features.

Furthermore, with this system, the C-shaped couplings consist of clamps with an adjustable orifice, that allow the bone fixation screw to be fitted in various positions, thus avoiding to a great extent the need to bend the rod and eliminating risks of cold-working, stress, etc. The above means that the rods can be smooth and unthreaded, insofar as threading leads to stress-raisers, and without the need for a lot of nuts, in such a way that good vertebral fixation can be achieved with a reduced number of parts.

On the other hand the coupling and the rod are fitted with an open swivel that allows for the orientation of one part in relation to another, the rigorous 90° of other systems is avoided, as is torsion and additional load on the rod, increasing the life of the implant, eliminating surgical problems, definitively cutting down intervention time and decreasing the risks of complications.

The screws used are hollow, like plugs, so that when a pin is passed through the middle they expand at the end inserted into the bone, thus increasing their resistance to pull out, making it unnecessary to traverse the two corticals of the bone and eliminating the risk of damage to blood vessels, tendons, etc.

The hooks used for this system are joined to the coupling in the same way as the screws, i.e. the threaded tail is inserted into the adjustable orifice of the coupling, being attached behind by the locknuts, essentially using two types of hook, laminar and pedicle, depending on where they are to be fitted.

To sum up, this system helps to reduce surgical intervention time and, therefore, the exposure of the opened body to the atmosphere, and simplifying to a great extent the instruments required.

In continuation, in order to contribute to a better understanding of this descriptive report, and forming an integral part hereof, a series of figures are attached, for illustrative purposes but not limitative, representing as follows:

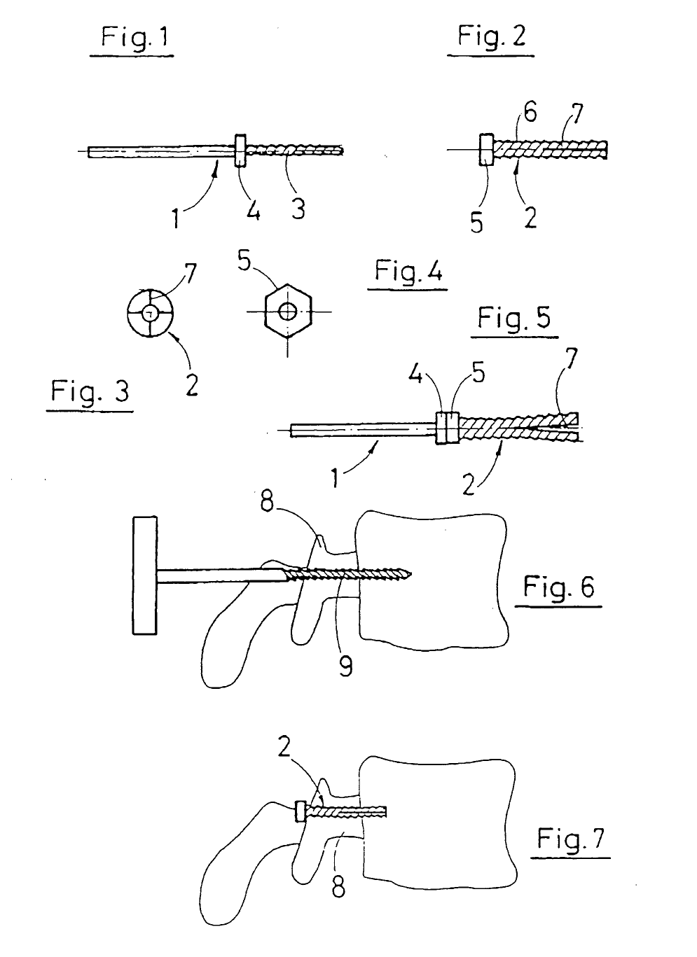

FIG. 1 shows a screw for forming a part of the device for carrying out the method of the present invention.

FIGS. 2, 3 and 4 are different projections of the plug forming a part of the device according to the invention.

FIG. 5 is a view of the device comprising the screw and the plug.

FIGS. 6, 7 and 8 show different stages of the system in which the technique for fitting the device is shown.

FIG. 9 corresponds to a system of the prior art, said figure showing the risks involved with the use of a conventional sized pedicular screw.

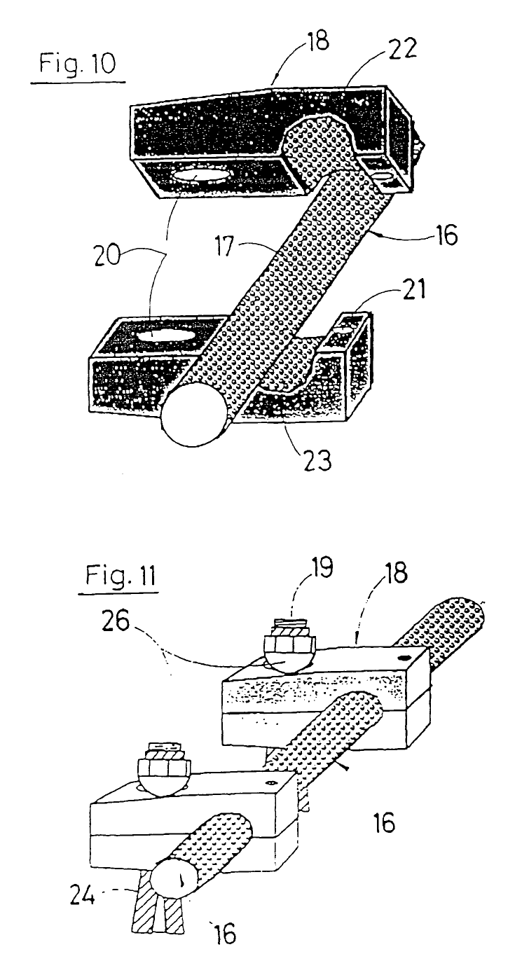

FIG. 10 is an exploded perspective view of the components of the system, being assembled according to an alternative embodiment consisting of a bare and two gripper arms forming the clamp.

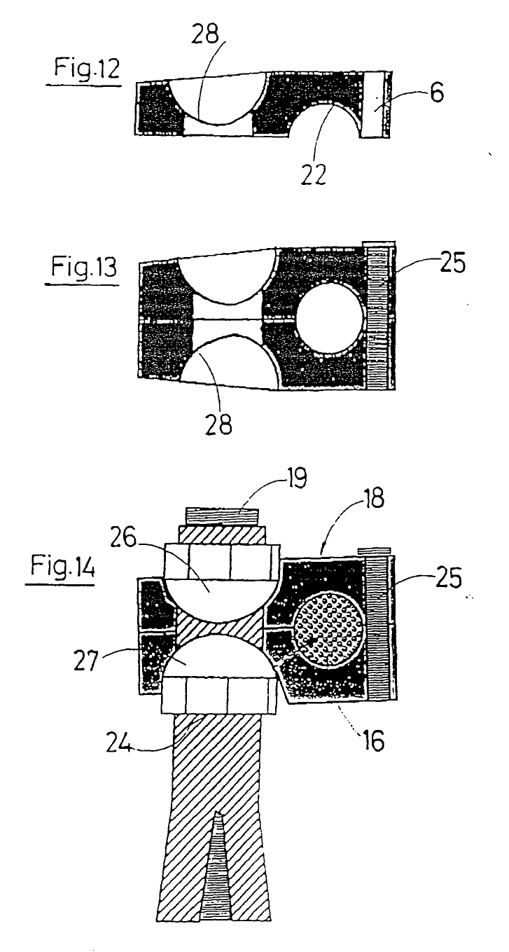

FIG. 11 is a perspective view showing vertebral intersegmentary attachment system according to FIG. 14.

FIG. 12 is a sectional view of a gripper arm.

FIG. 13 is a sectional view of two assembled gripper arms forming a clamp.

FIG. 14 shows the plug and screw passing through the two gripper arms in form of a clamp which in turn holds a connecting bar.

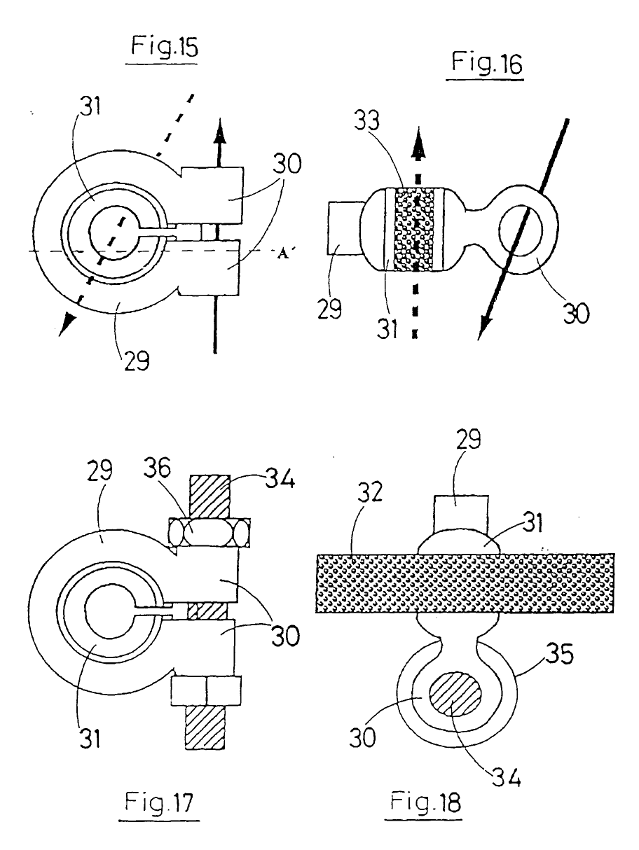

FIG. 15 is a plan view from below of a clamp according to an alternative embodiment.

FIG. 16 is a sectional view of the clamp along the line A-A′ of FIG. 15.

FIG. 17 is a similar view to that of FIG. 15, in which the clamp includes the screw to be inserted in the pedicular plug.

FIG. 18 is a schematic view of the clamp with the fixing bar and the cylindrical member with the screw and the pedicular plug.

FIG. 19 is a cross section of a practical embodiment with an expansion screw.

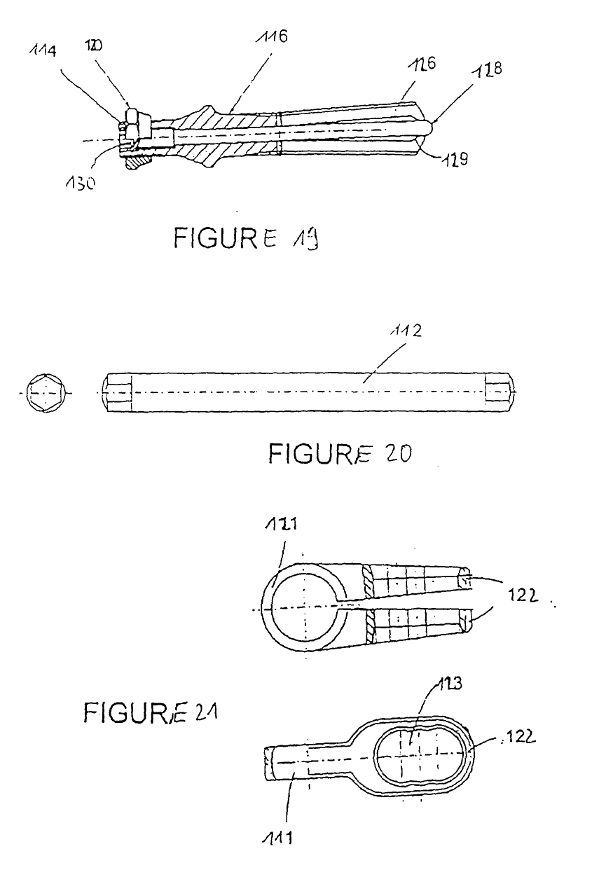

FIG. 20 is an elevation view of the system rod.

FIG. 21 is an elevation and plan view of a coupling or connector with adjustable orifice.

FIG. 22 is an elevation views and profile view of a pedicle hook.

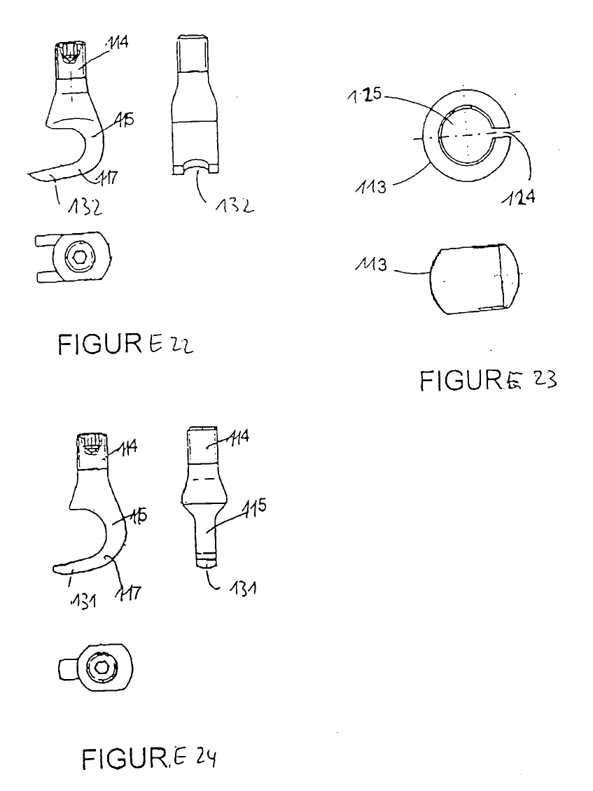

FIG. 23 is an elevation view and plan view of a multidirectional swivel.

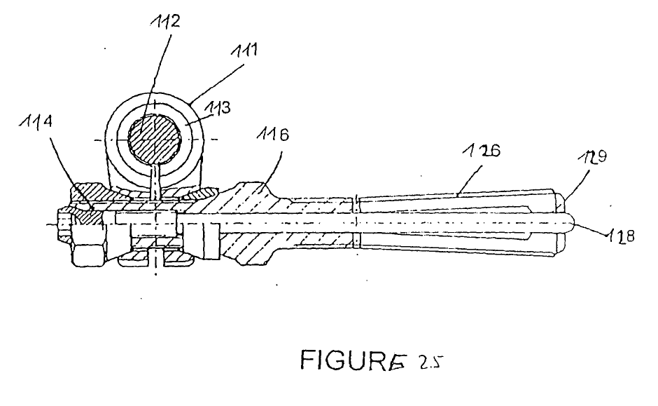

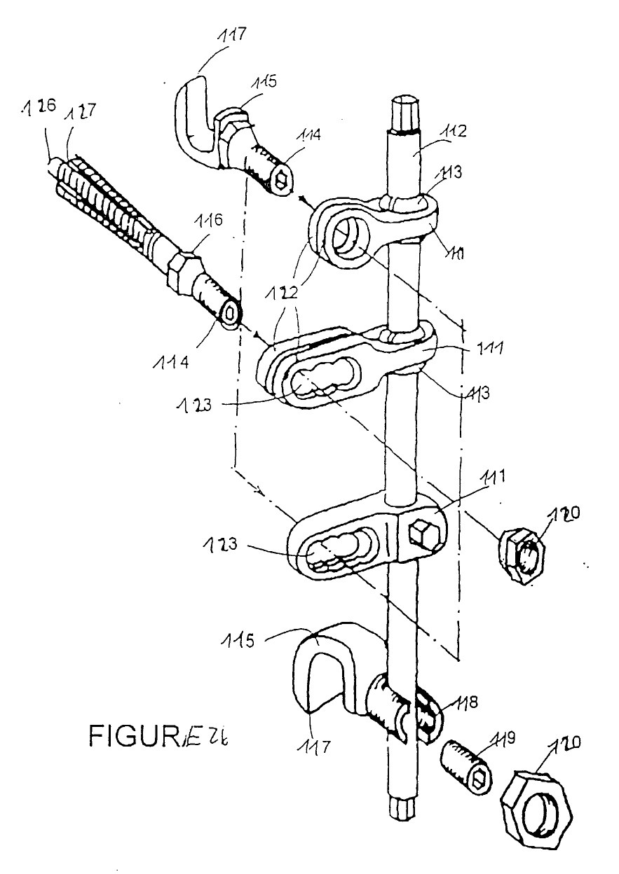

FIG. 24 is an elevation view and profile view of a laminar hook.

FIG. 25 is a cross section of an assembly of the system, object of the present invention.

FIG. 26 is a perspective drawing of a practical embodiment of the vertebral fixation system, object of the present invention.

With reference to FIGS. 1 to 9, there is to be seen a device designed for vertebral attachment exclusively with transpedicular support, comprising a screw which may be of any biocompatible and mechanically stable material. Preferred embodiments of the invention comprise a screw made of steel, titanium or any alloy designated in general by 1, and a plug 2 of a biocompatible and mechanically stable material, which may, in accordance with a preferred embodiment of the invention, be a silastic or any other polymer of similar mechanical properties and biocompatibility, or of any metal or metal alloy.

The screw 1 has a threaded portion 3, limited by a radial shoulder 4. The plug 2 has a radial widening 5 to prevent its being inserted too far into the pedicle, and the shank of said plug is provided with a thread 6. The widened portion 5 may be hexagonal in shape to allow it to engage a hollow screw driver, allowing it to be manipulated.

Said plug 2 may be of the type having longitudinally extending slits 7 to promote the expansion thereof. In an alternative embodiment, a plug 2 may be used which has no longitudinally extending slits 7 and cannot be expanded accordingly.

In the method according to the present invention, there is comprised a first step in which the pedicle 8 is drilled, for example with a twist drill 9, which may have any desirable diameter, e.g. a diameter of 0.4 cm, as shown in FIG. 6.

In a second step of the method according to the invention, as shown in FIG. 7, the plug, e.g. the silastic plug 2, is inserted into the pedicle 8 by screwing it into the bore formed with the (twist) drill.

In the case that a plug having no longitudinally extending slits is used, the insertion of the plug with one of its ends into the hole formed in the pedicle establishes a firm anchorage of the plug in the pedicle.

Finally, in an optional third step, the threaded portion 3 of the screw is inserted into the plug 2 and screwed into the bore of said plug, thereby expanding a second end thereof. Thereby, an even better anchorage of the plug in the pedicle may be obtained. This is, however, not a necessary feature of the invention.

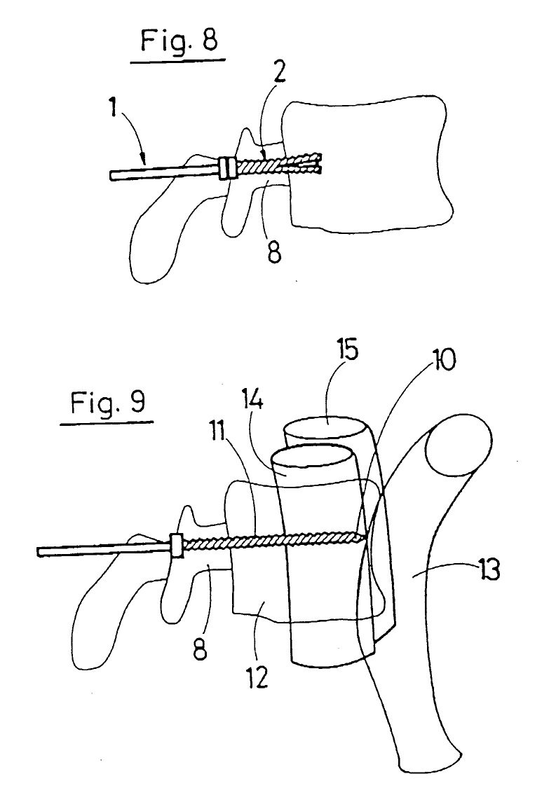

In FIG. 8, the advantages provided by the improvements of this embodiment of the invention of the prior art (which is shown in FIG. 9) are shown: the tip 1 of the conventional pedicular screw 11 is shown to be in a position to extend beyond the anterior cortical layer of the vertebrae 12 and perforate viscera or blood vessels (intestine 13, aorta 14, vena cava 15).

With reference to FIGS. 10 to 14 there can be seen rods 16 formed of the same material as the plug or of a different material which, however, must also be biocompatible. The rods have, in preferred embodiments, a diameter of 4.6 to 6 mm and length ranging from 3 to 25 cm. The rods 16 are cylindrical, and the surface thereof, in a preferred embodiment, may be finished to prevent slipping, for example in 0.4 mm high diamond points 17.

The clamps 18 are used to attach the rods 16 to the pedicular screws 19. They are formed by two symmetrical members, each of them of rectangular form, having two holes 20 and 21 and a groove 22. The groove runs from top to bottom, and the surface may, in a preferred embodiment, also be diamond point finished 23 for holding the rod 16 and preventing a slippage thereof. The larger hole 20 has the same bore as the diameter of the pedicular plug 24 which it should house in the arrangement when the system is assembled. The smaller hole 21 located on the opposition side to the larger one, is for housing a small screw 25 which joins the two gripper arms together, helping firmly to hold the rod 16 there between, preventing a slippage thereof.

The attachment of the pedicular plugs 24 to the clamps 18 is consolidated by usual means which may be selected by a skilled person, preferably by two round headed blind nuts 26 and 27 screwed on the plug and which hold the two gripper arms between them, after the plug had been inserted in the corresponding hole in the gripper arms. The thickness of the gripper arms is reduced a this level, and the holes for the screw are also spherically hollowed out 28 to allow any angle of attack of the plug against the gripper arm.

With reference to FIGS. 15 to 18, there is seen an alternative embodiment of clamp connection means applicable in the vertebral fusion system a way of a pedicular plug and screw and a connecting rod. Such means or clamps are constituted by two parts which are designated as 2 and 3, respectively. Part 29 is a cylindrical circular clamp member open at one of the sides thereof and housing therein a ball joint 31 which is also open at one side thereof.

Part 30 constitutes the hollow cylinders, each attached to one of the open ends of the circular member 29.

The attachment rod 32 is inserted to the circular member 29 and the ball joint 31. For the ball joint to grip the rod 32 firmly, the internal surface thereof for engaging the rod 32 may be provided, in a preferred embodiment thereof, with a diamond point finish 33.

A screw 34 is inserted through the two portions of the cylindrical member and is then inserted in the pedicular plug or directly in the pedicle and vertebra. Once the screw has been inserted, the system is blocked with a nut 36.

In FIGS. 15 and 16, the dotted arrows show the passage of the rod, and the solid arrow shows the passage of the pedicular screw.

As can be seen in the FIGS. 19 to 26, various parts are distinguished as part of the system, on the one hand the couplings and connectors 111 with their swivels 113, a rod 112, a screw 116 with a tail 114 to screw into the coupling, and also hooks 115 with their tails 114 and their respective fingers 117 to be attached to the vertebra, or this hook to be connected directly to the rod 112 by means of a split head 118, with closure using a setscrew 119 and a nut 120.

These couplings or connectors 111, which are very similar to the embodiment described in connection with the FIGS. 15 to 18, essentially consist of an annular part 121, with two clamping elements 122, in such a way that they allow for a certain flexibility of the body, given that they naturally adopt an open position. On the other hand these clamps have a transverse adjustable orifice 123, an orifice that can be circular, as required, for the precise positioning of the tail 114 of the vertebral attachment element.

The annular body 121 houses the open swivel 113 in such a way that with the clamp in its natural position the swivel turns freely in its housing with three degrees of freedom, with the exterior radius of the swivel 113 slightly lower than the inside of the coupling 111 ring 121, both being concentric radii.

The swivel consists of a body 113 opened by a slot 124, with a passing orifice 125, through which the system rod 112 can pass. The exterior surface of this swivel has a rough finish so that when it is pressed onto the clamp 122 and the opening slot 124 is closed, then the contact between the exterior surface of the swivel 113 and the interior surface of the annular body 121 of the coupling 111 prevents all movement.

One of the main elements of this system is the pedicle screw 116, consisting of a tail 114 for connection to the coupling 111 and the rod 112, and a body with slots 127 that start half way along the threaded length forming slits 126, with the body of the screw, externally threaded, hollow and smooth on the inside, allowing it to house a pin 128 which slides against an inclined plane 129, forcing it to open pressing against the sponginess of the vertebral body, in such a way that the diameter of the lower third of the expansion screw 116 increases progressively towards the end, until it reaches its maximum at a point between 20 and 30% when completely expanded.

The tail 114 of the screw 116 has an interior thread with which to pick up the head 130 of the pin 128, which is machine on the interior, like an Allen-screw, allowing it to be attached to the screw 116.

Other fixation elements that are used for the coupling of the system to the vertebra are hooks 115, fitted with a tail 114 for their upper connection to the coupling 111 and a vertebra coupling finger 117, this finger being smooth 131 in the case of laminar hooks or concave 132 for pedicle hooks.

Another of the hooks 115 used with this system is a hook with an open tail 118, inside of which the rod 112 is fitted, subsequently locked into place by a setscrew 119 on the inside and a locknut 120 on the outside.

Application and operation are simple. The system is designed for dorsolumbar and lumbosacral pathologies, such as degenerative pathologies, osteoporosis, tumors, fractures, re-interventions on the sacral spine and spinal deformities (scoliosis).

The approach path is posterior, using screws 116 and hooks 115 according to the level and/or the pathology concerned.

Firstly the pedicle is located, for the use of the expansion screw 116, a bit and tapper are used to make a hole in the pedicle, then the screw 116 is inserted in the hole in the vertebra with the pin 128 inside the hollow body of the screw 116, but not fully housed. This assembly is inserted into the hole that has been made. All of the screws 116 required by the system are inserted in this way.

At the same time an assembly is put together with the open swivel 113 and the coupling 111, by means of the annular body 121. These couplings or connectors 111 are assembled on the rod 112, this is done by passing the rod through the swivel orifice 125.

Once the respective couplings 111 have been positioned on the rod, aligned with the inserted screws 116, the threaded tails 114 of the screws 116 are inserted through the adjustable orifices 123 of the coupling clamp 122, in the most adequate position for the assembly of the system. In those cases in which this coupling does not have adjustable holes, it will have to be inserted through normal circular orifices.

In this position the couplings 111 are orientated towards the tails 114 of the screws 116, to this end the multidirectional property of the open swivels 113 is used by merely turning the coupling around the rod 112, allowing for a few, for example 3, degrees of freedom in this articulation.

Once all of the parts have been coupled, the nut 120 is attached and locked onto each of the tails 114 of the different screws. This nut is then made up, thus closing the clamp 122, which in turn closes the opening slot 124 of the swivel 113, thus attaching the system, given that, on the one hand, the swivel is fixed to the exterior surface of the rod 112, and on the other the coupling 111, by means of the rough contact against the interior surface of the annular body 121, eliminates any possibility of swivel 113 movement.

When this task has been concluded, the pin 128 is inserted as far as it will go, using a screwdriver, thus forcing the screw 116 to expand. To do this the screwdriver is applied to the head of the pin 130 which is housed inside the tail 114 of the screw. As the point of the pin 128 is made up it meets the interior taper 129 of the smooth interior hollow of the screw, as it continues advancing this pin opens out the end of the screw, thanks to the slots 127 forcing the slits 126 of the screw to expand against the sponginess of the vertebra, until it reaches the stop and the screw is completely expanded.

For those cases in which the use of hooks 115 is indicated, then the grounding of the hook 115 is worked with a suitable instrument, coupling it to the finger 117, either laminar in cases of laminar fingers 131 or gripping the pedicle through the concave shape 132 of the finger 117, depending on which hook 115 is used and the procedure.

Subsequently the coupling 111 connection is carried out in a similar way to the assembly of the expansion screws 116.

The fitting of open tail 118 hooks 115 is somewhat similar, with the rod 112 being passed through them, and with the assembly finally being closed in by a setscrew 119 on the inside and a locknut 120 on the outside.

Having sufficiently described the nature of the present invention, as well as the means for it to be put into practice, it only remains for us to add that the introduction of changes of form, both overall and in terms of the different parts that make up the invention, as well as in terms of both materials and the layout thereof, is possible, always insofar as such alterations do not substantially vary the characteristics of the invention that is distinguished in the following claims.

1. A method for transpedicular vertebral attachment comprising the steps of
drilling a pedicle with a drill as for example a twist drill in order to form a hole in the pedicle;
removing the drill from the hole thus formed in the pedicle;
screwing a plug into the hole in the pedicle thus formed by the twist drill; and
thereby establishing an anchorage of the plug in the pedicle.

2. The method according to claim 1, wherein the plug has a first end and a second end and has an opening at the first end thereof which is not inserted into said hole formed in the pedicle, and said plug has a tubular stem being provided with equidistantly spaced radial slits extending lengthwise along the stem at the second end; and a threaded portion of a screw is inserted into the opening of the plug provided at the first end thereof, whereby the plug is radially expanded at its second end, whereby an anchorage of the plug is established in the pedicle.

3. The method according to claim 1, further comprising the step of using a twist drill of 0.4 cm size.

4. The method according to claim 2, further comprising the step of using a screw which is made of steel, titanium or an alloy.

5. The method according to claim 2, further comprising the steps of
providing a radially widened portion on the plug;
providing a radial shoulder on the screw, the threaded portion of the screw extending from an end of the screw to the radial shoulder, the threaded portion of the screw being slightly longer than a length of the pedicle; and
stopping inserting the screw into the hole in the plug by engagement of the radial shoulder with the radially widened portion of the plug.

6. The method according to claim 1, further comprising the steps of
using a polymer or a metal or an alloy for the plug, the plug being biocompatible with the pedicle, the plug having a radially widened portion, said tubular stem of the plug extending from an end thereof to the radially widened portion, the tubular stem preferably having a length of 3.1 cm and a diameter of 0.5 cm, the radially widened portion forming a hexagonal head preferably having a diagonal measurement of 0.8 cm; and
engaging the hexagonal head with a tool for manipulation thereof.

7. The method according to claim 2, further comprising the step of enlarging the radial slits to thereby widen the end of the plug which is screwed into the hole formed in the pedicle during the step of expanding the plug, the tubular stem having a first circumference adjacent the radially widened portion and having a second circumference at the end which is screwed into the hole in the pedicle, the second circumference being larger than the first circumference after the step of enlarging.

8. The method according to claim 1, further comprising the steps of using silastic as the material of the plug, the plug being biocompatible with the pedicle, the plug having a radially widened portion and a tubular stem extending from an end thereof to the radially widened portion, the tubular stem preferably having a length of 3.1 cm and a diameter of 0.5 cm, and the radially widened portion forming a hexagonal head preferably having a diagonal measurement of 0.8 cm; and engaging the hexagonal head with a tool for manipulation thereof.

9. The method according to claim 8, further comprising the step of enlarging the radial slits to thereby widen the end of the plug which is screwed into the hole formed in the pedicle during the step of expanding the plug, the tubular stem having a first circumference adjacent the radially widened portion and having a second circumference at the end which is screwed into the hole in the pedicle, the second circumference being larger than the first circumference after the step of enlarging.

10. A system for transpedicular vertebral attachment comprising
a drill, preferably a twist drill, for forming a hole in the pedicle;
a plug being insertable, with one of its ends, into the hole in the pedicle formed by the drill, whereby an anchorage of the plug in the pedicle may be obtained.

11. The system for transpedicular vertebral attachment according to claim 10, wherein the plug comprises a first end and a second end and has, at its first end, an opening provided therein, the second end of the plug being insertable into the hole in the pedicle formed by the drill and having a tubular stem being provided with equidistantly spaced radial slits extending lengthwise along the stem; and the system comprises means for expanding the second end of the plug to thereby enlarge the second end to form an anchorage of the plug in the pedicle, the means for expanding comprising a screw which insertable, preferably may be screwed, into the opening in the first end of the plug.

12. The system for transpedicular vertebral attachment according to claim 10, wherein the drill has a size of 0.4 cm.

13. The system for transpedicular vertebral attachment according to claim 11, wherein the screw is made of steel, titanium or an alloy.

14. The system for transpedicular vertebral attachment according to claim 11, wherein the plug has a radially widened portion and the screw has a radial shoulder, the screw having a threaded portion which extends from an end of the screw to the radial shoulder, the threaded portion of the screw being slightly longer than a length of the pedicle, the radial shoulder of the screw being adapted to engage the radially widened portion of the plug to be able to stop an insertion of the screw into the opening of the plug.

15. The system for transpedicular vertebral attachment according to claim 10, wherein the plug is made of one material selected from a polymer, a metal or an alloy and is biocompatible with the pedicle the plug having a radially widened portion, the tubular stem extending from the second end thereof to the radially widened portion, the tubular stem preferably having a length of 3.1 cm and a diameter of 0.5 cm, and the radially widened portion forming a hexagonal head preferably having a diagonal measurement of 0.8 cm.

16. The system for transpedicular vertebral attachment according to claim 11, wherein the radial slits are enlarged by the means for expanding to thereby widen the second end of the plug, the tubular stem having a first circumference adjacent the radially widened portion and having a second circumference at the end which is screwed into the hole in the pedicle, the second circumference being larger than the first circumference after the means for expanding enlarges the second end of the plug to form an anchorage in the pedicle.

17. The system for transpedicular vertebral attachment according to claim 11, wherein the plug has a radially widened portion and the tubular stem extending from the second end thereof to the radially widened portion, the means for expanding enlarges the radial slits to thereby widen the second end of the plug, the tubular stem having a first circumference adjacent the radially widened portion and having a second circumference at the end which is screwed into the hole in the pedicle, the second circumference being larger than the first circumference after the means for expanding enlarges the second end of the plug to form an anchorage in the pedicle.

18. The system for transpedicular vertebral attachment according to claim 11, wherein the plug is made of silastic and is biocompatible with the pedicle, the plug having a radially widened portion, the tubular stem extending from the second end thereof to the radially widened portion, the tubular stem preferably having a length of 3.1 cm and a diameter of 0.5 cm, and the radially widened portion forming a hexagonal head preferably having a diagonal measurement of 0.8 cm.

19. The system for transpedicular vertebral attachment according to claim 18, wherein the radial slits are enlarged by the means for expanding to thereby widen the second end of the plug, the tubular stem having a first circumference adjacent the radially widened portion and a having second circumference at the end which is screwed into the hole in the pedicle, the second circumference being larger than the first circumference after the means for expanding enlarges the second end of the plug to form an anchorage in the pedicle.

20. A device for connecting an attachment rod and a pedicular plug in a transpedicular vertebral attachment system, said device comprising:
a generally cylindrical, C-shaped member having two ends which form an opening therebetween, the C-shaped member further having a centrally disposed opening, a ball joint being housed in said centrally disposed opening of the C-shaped member, the attachment rod being housed in the ball joint, the ball joint being in direct engagement with the attachment rod;
a pair of hollow cylinders, one of the cylinders being attached to each end of the C-shaped members, a screw being received in both of the hollow cylinders, the screw being received insertable in the pedicular plug; and
a means, preferably a nut, for locking the screw in position in the pair of hollow cylinders.

21. The connecting device according to claim 18, wherein the ball joint has a diamond point finish in direct contact with the attachment rod.

22. A dorsolumbar and lumbosacral vertebral fixation system, wherein the system consists of one or various connectors or couplings (111), a rod (112), a transversal traction device and means of vertebral fixation, with assembly carried out by the attaching the tail (114) of the vertebral element—coupling (111)—rod (112), the first assembly stage of the system being the introduction of the fixation elements, either to the pedicle or the vertebral laminae, a second stage of the insertion of the rod (112) through the connectors (111), and a third stage in which the connectors are connected to the tails (114) of the fixation elements by means of locknuts (120).

23. The dorsolumbar and lumbosacral vertebral fixation system, as in claim 22, wherein the connectors (111) are made up of an annular body (121) and two clamp elements (122) with the insertion of an open swivel (113) inside the annular body (121).

24. The dorsolumbar and lumbosacral vertebral fixation system, as in claim 22, wherein, with the clamp (122) open in its natural position, the swivel (113) turns freely in its housing, preferably with three degrees of freedom, in a radius exterior to the swivel (113) slightly smaller than the inside of the ring (121) of the clamp, both being concentric radii.

25. The dorsolumbar and lumbosacral vertebral fixation system, as in claim 22, wherein the open clamp (122) has a transversal circular orifice into which the tail (114) of the fixation elements is inserted.

26. The dorsolumbar and lumbosacral vertebral fixation system, as in claim 22, wherein the open clamp (122) has an adjustable transversal orifice (123) that allows for different tail (114) positions of the vertebral fixation elements.

27. The dorsolumbar and lumbosacral vertebral fixation system, as in claim 22, wherein the open swivel (113) is hollow with a circular shape (125) through which the rod (112) passes.

28. The dorsolumbar and lumbosacral vertebral fixation system, as in claim 22, wherein the exterior surface of the swivel (113) has a rough finish, which allows for better contact between surfaces when tightened.

29. The dorsolumbar and lumbosacral vertebral fixation system, as in claim 22, wherein the screw tightness of the tail (114) of the vertebral fixation element on the clamp (122), closes the body of the clamp which, in turn, closes the swivel (113) opening-slot (124), thus tightening onto the previously oriented rod (112), fixing it in place.

30. The dorsolumbar and lumbosacral vertebral fixation system, as in claim 22, wherein, as a vertebral fixation element, an expansion screw (116) is used, this being a hollow pedicle screw, smooth on the inside, through which a pin (128) is passed.

31. The dorsolumbar and lumbosacral vertebral fixation system, as in claim 22 wherein the screw head has an interior thread in order to house, threaded in, the Allen-type screw of the screw head (130) of the pin (128).

32. The dorsolumbar and lumbosacral vertebral fixation system, as in claim 22, wherein the expansion screw consists of lengthways slots (127), which start towards the middle of its threaded length, and which are opened by fully inserting the pin (128).

33. The dorsolumbar and lumbosacral vertebral fixation system, as in claims 22, wherein the diameter of the lower third of the expansion screw (116), when the pin is fully inserted, progressively increases towards the end, until it reaches its maximum at the tip, between 20 and 30% when completely expanded.

34. The dorsolumbar and lumbosacral vertebral fixation system, as in claim 22, wherein the expansion screw (116) is used in cases of osteoporosis vertebrae, re-interventions and for the sacral vertebrae, in order not to penetrate the anterior cortical layer.

35. The dorsolumbar and lumbosacral vertebral fixation system, as in claim 22, wherein prior to the insertion of the expansion screw (116) the bone is tapered to the same thread as the external thread of the expansion screw.

36. The dorsolumbar and lumbosacral vertebral fixation system, as in claim 22, wherein the expansion screw (116) consists of a lengthways interior hollow conduit, with an internal taper (129) towards the end, in such a way that when the pin is inserted (128), without making up the head (130) the tip of this pin reaches the said inclined plane (129).

37. The dorsolumbar and lumbosacral vertebral fixation system, as in claim 22, wherein, when the head (130) of the pin (128) is made up on the tail (114) of the expansion screw (116) the tip of the pin (128) opens the internal taper (129) forcing the slots (127) of the screw (126) to open out, expanding the screw against the sponginess of the vertebral body.

38. The dorsolumbar and lumbosacral vertebral fixation system, as in claim 22, wherein, as a fixation element a laminar (131) hook (115) is used, which couples onto the vertebral lamina by means of a hook finger and is screwed to the coupling (111) at the top.

39. The dorsolumbar and lumbosacral vertebral fixation system, as in claim 22, wherein, as a vertebral fixation element a pedicle (132) hook (115) is used, which couples onto the pedicle of the vertebra by means of a concave shape on the finger of the hook, and is screwed to the coupling (111) at the top.

40. The dorsolumbar and lumbosacral vertebral fixation system, as in claim 22, wherein, as a fixation element an open tail (118) hook (115) is used, as a top connection directly to the rod (112), being closed and attached by means of a locknut (120) and a locking setscrew (119).
